(19) United States
(12) Patent Application Publication
(10) Pub. No.: US 2023/0165533 A1
(43) Pub. Date: Jun. 1, 2023

(54) HEALTH MANAGEMENT DEVICE, HEALTH MANAGEMENT METHOD, AND HEALTH MANAGEMENT SYSTEM

(71) Applicant: TOYOTA JIDOSHA KABUSHIKI KAISHA, Toyota-shi (JP)

(72) Inventors: Ryo MURAKAMI, Susono-shi (JP); Shinnosuke SHIMOKAWA, Susono-shi (JP); Junichi MORI, Nagakute-shi (JP); Sokfan YEE, Toyota-shi (JP); Kensaku SHIBATA, Toyota-shi (JP); Yuta MANIWA, Susono-shi (JP)

(21) Appl. No.: 18/045,188

(22) Filed: Oct. 10, 2022

(30) Foreign Application Priority Data
Nov. 26, 2021 (JP) 2021-192160

(51) Int. Cl. A61B 5/00

(52) U.S. Cl. CPC A61B 5/6893; A61B 5/742; A61B 5/7475

(57) ABSTRACT

A health management device includes: an acquisition unit configured to acquire a weight of an occupant detected by a detector provided in a seat of a vehicle that transports the occupant or a value detectable using the weight as a characteristic value associated with a physical body of the occupant; and a display information generating unit configured to generate display information which is displayed on a display device which is visible to an occupant when the occupant sits on the seat based on the characteristic value, the display information varying according to a difference between the characteristic value detected at the timing at which the occupant sits on the seat and the characteristic value of the occupant detected in the past.

### CROSS-REFERENCE TO RELATED APPLICATION

This application claims priority to Japanese Patent Application No. 2021-192160 filed on Nov. 26, 2021, incorporated herein by reference in its entirety.

### BACKGROUND

### 1. Technical Field

The disclosure relates to a health management device, a health management system, and a health management method.

### 2. Description of Related Art

In the related art, a weighable chair that can accurately and easily measure a weight of a person with a natural posture when the person sits on a regular chair is known (for example, see Japanese Unexamined Patent Application Publication No. 9-105663 (JP 9-105663 A)).

### SUMMARY

Recently, people have become increasingly aware of their health with the increase in health consciousness. However, the viewpoint of promoting health using a vehicle has not been sufficiently studied in the related art. In the technique described in JP 9-105663 A, a weight indicating a degree of healthiness can be measured by causing a person to sit on a chair, but there is room for improvement in view of using an opportunity when boarding a vehicle for health promotion of an occupant.

The disclosure provides a health management device, a health management method, and a health management system that can achieve health promotion for an occupant using an opportunity when boarding a vehicle.

The present disclosure is summarized as follows.

(1) According to an aspect of the disclosure, there is provided a health management device including: an acquisition unit configured to acquire a weight of an occupant detected by a detector provided in a seat of a vehicle that transports the occupant or a value detectable using the weight as a characteristic value associated with a physical body of the occupant; and a display information generating unit configured to generate display information which is displayed on a display device which is visible to an occupant when the occupant sits on the seat based on the characteristic value, the display information varying according to a difference between the characteristic value detected at the timing at which the occupant sits on the seat and the characteristic value of the occupant detected in the past.

(2) The health management device according to (1) may further include a discount rate calculating unit configured to calculate a discount rate of a fare for allowing an occupant to use the vehicle based on the difference in characteristic value, and the display information generating unit may be configured to generate the display information indicating the discount rate.

(3) In the health management device according to (2), the discount rate calculating unit may be configured to calculate the discount rate such that the discount rate becomes larger as the characteristic value becomes closer to a suitable value for the occupant.

(4) The health management device according to (1) may further include: a target walking distance calculating unit configured to calculate a target walking distance of an occupant based on the difference in characteristic value; and a recommended alighting place determining unit configured to determine a recommended alighting place which is the target walking distance prior to a scheduled alighting place based on the target walking distance and the scheduled alighting place at which the occupant is scheduled to alight, and the display information generating unit may be configured to generate display information indicating the recommended alighting place.

(5) In the health management device according to (4), the target walking distance calculating unit may be configured to calculate the target walking distance such that the target walking distance becomes smaller as the characteristic value becomes closer to a suitable value for the occupant.

(6) The health management device according to (4) or (5) may further include a discount rate calculating unit configured to calculate a discount rate of a fare for allowing an occupant to use the vehicle based on the target walking distance when an agreement with alighting at the recommended alighting place is acquired from the occupant, and the display information generating unit may be configured to generate display information indicating the discount rate.

(7) In the health management device according to (6), the discount rate calculating unit may be configured to calculate the discount rate such that the discount rate becomes larger as the target walking distance becomes larger.

(8) The health management device according to any one of (4) to (7) may further include a notification unit configured to notify a control system that automatically controls the vehicle or a notification device that notifies a driver of the vehicle of a stop place of the recommended alighting place when an agreement with alighting at the recommended alighting place is acquired from the occupant.

(9) In the health management device according to (3) or (5), the characteristic value may be a weight, and the health management device may further include a suitable weight calculating unit configured to calculate a suitable weight as the suitable value based on a height of the occupant.

(10) In the health management device according to any one of (1) to (9), the display device may be provided in a front end portion of a bearing surface of the seat.

(11) The health management device according to any one of (1) to (10) may further include a display processing unit configured to display the display information on the display device provided in the seat or in the vicinity of the seat.

(12) In the health management device according to any one of (1) to (9), the display device may be provided in a mobile terminal that is carried by an occupant, and the health management device may further include a transmission unit configured to transmit the display information to the mobile terminal.

(13) The health management device according to any one of (1) to (12) may further include an authentication unit configured to authenticate an occupant based on authentication information, and the display information generating unit may be configured to generate the display information which varies according to the difference in characteristic value of the occupant of which authentication has succeeded.

(14) The health management device according to (13) may further include a memory configured to store the characteristic value, the characteristic value may be stored in the memory in correlation with the authentication information, and the acquisition unit may be configured to acquire the characteristic value detected in the past from the memory based on the authentication information.

(15) In the health management device according to (13) or (14), the authentication information may be acquired from an input device to which an occupant is able to input information and which is provided in the vicinity of the seat or a mobile terminal that is carried by the occupant.

(16) In the health management device according to any one of (1) to (15), the characteristic value may be at least one of a weight, a body fat percentage, a visceral fat level, a subcutaneous fat percentage, a basal metabolic rate, a skeletal muscle percentage, a bone mass, a muscle mass, a BMI, and an FFMI.

(17) According to another aspect of the disclosure, there is provided a health management method including: a step of acquiring a weight of an occupant detected by a detector provided in a seat of a vehicle that transports the occupant or a value detectable using the weight as a characteristic value associated with a physical body of the occupant; and a step of generating display information which is displayed on a display device which is visible to an occupant when the occupant sits on the seat based on the characteristic value, the display information varying according to a difference between the characteristic value detected at the timing at which the occupant sits on the seat and the characteristic value of the occupant detected in the past.

(18) According to another aspect of the disclosure, there is provided a health management system in which a health management device and a mobile terminal are connected in a radio communication manner, wherein the health management device includes: an acquisition unit configured to acquire a weight of an occupant detected by a detector provided in a seat of a vehicle that transports the occupant or a value detectable using the weight as a characteristic value associated with a physical body of the occupant; a display information generating unit configured to generate display information which is displayed on a display device which is visible to an occupant when the occupant sits on the seat based on the characteristic value, the display information varying according to a difference between the characteristic value detected at the timing at which the occupant sits on the seat and the characteristic value of the occupant detected in the past; and a transmission unit configured to transmit the display information to the mobile terminal, and wherein the mobile terminal includes a reception unit configured to receive the display information and a display processing unit configured to display the display information on a display unit provided in the mobile terminal.

According to the disclosure, it is possible to provide a health management device, a health management method, and a health management system that can achieve health promotion for an occupant using an opportunity when boarding a vehicle.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a diagram schematically illustrating a configuration of a health management system according to a first embodiment of the disclosure;

FIG. 2 is a diagram schematically illustrating a configuration of a seat on which an occupant sits and which is provided in a vehicle;

FIG. 3 is a diagram schematically illustrating a configuration of a seat on which an occupant sits and which is provided in a vehicle;

FIG. 4 is a diagram schematically illustrating functional blocks of a processor of a control device;

FIG. 5 is a flowchart illustrating a routine which is performed by the processor of the control device;

FIG. 6 is a flowchart illustrating another routine which is performed by the processor of the control device;

FIG. 7 is a diagram schematically illustrating a display screen of a display device and illustrating a situation in which display information is displayed on the display screen;

FIG. 8 is a diagram schematically illustrating a display screen of a display device and illustrating a situation in which display information is displayed on the display screen;

FIG. 9 is a diagram schematically illustrating a configuration of a health management system according to a second embodiment of the disclosure;

FIG. 10 is a diagram schematically illustrating functional blocks of a processor of a control device;

FIG. 11 is a diagram schematically illustrating functional blocks of a control unit provided in a mobile terminal; and

FIG. 12 is a sequence diagram illustrating a routine which is performed by the mobile terminal and the control device.

### DETAILED DESCRIPTION OF EMBODIMENTS

Hereinafter, some embodiments of the disclosure will be described with reference to the accompanying drawings. Such description is intended to merely exemplify preferred embodiments of the disclosure, but is not intended to restrict the disclosure to the embodiments. In the following description, the same elements will be referred to by the same reference signs.

### 1. First Embodiment

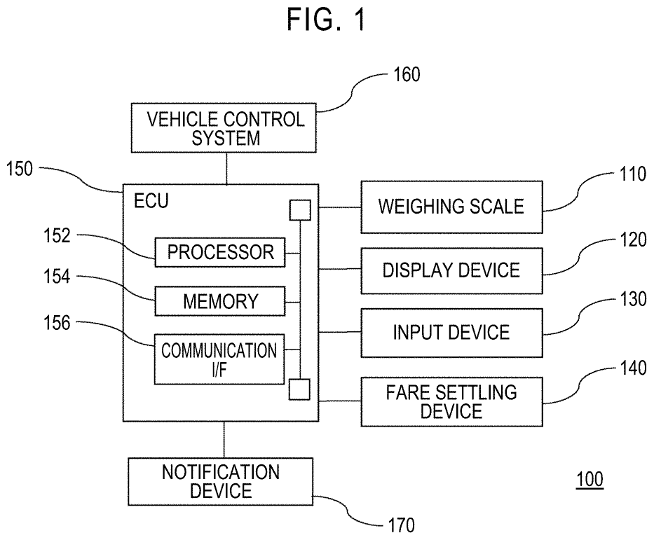

FIG. 1 is a diagram schematically illustrating a configuration of a health management system 100 according to a first embodiment of the disclosure. The health management system 100 is mounted in a vehicle that travels on a road and transports an occupant. In this embodiment, a regular bus is exemplified as the vehicle in which the health management system 100 is mounted. For example, the vehicle may be an automated driving bus that travels on a road based on predetermined commands and transports passengers, and may operate periodically in a smart city. The smart city is a sustainable city or district which has been proposed by the Ministry of Land, Infrastructure, and Transport and in which management (such as planning, maintenance, and administration and operation) is performed using new technology such as information and communication technology (ICT) and the whole optimization is achieved. The vehicle is not limited to an automated driving vehicle, but may be a vehicle that is manually driven.

The health management system 100 measures a weight or the like of an occupant in a vehicle and cares the occupant's health based on the measured weight or the like. The health management system 100 includes a weight scale 110, a display device 120, an input device 130, a fare settling device 140, a control device 150, a vehicle control system 160, and a notification device 170. The control device 150, the weight scale 110, the display device 120, the input device 130, the fare settling device 140, the vehicle control system 160, and the notification device 170 are connected to communicate with each other via an onboard network based on a standard such as controller area network (CAN) or Ethernet (registered trademark).

The weight scale 110 is a type of a detector provided in a seat of a vehicle that transports an occupant. The weight scale 110 detects an occupant's weight or a value that can be detected using the weight (for example, a body fat percentage, a visceral fat level, a subcutaneous fat percentage, a basal metabolic rate, a skeletal muscle percentage, a bone mass, a muscle mass, a body mass index (BMI), and a fat free mass index (FFMI)) as characteristic values associated with the occupant's physical body.

The display device 120 is constituted by, for example, a liquid crystal display (LCD) and displays various types of information such as an occupant's weight. The display device 120 displays display information which is generated by the control device 150.

The input device 130 is a device which is constituted by a touch sensor, a keyboard, or the like and to which an occupant can input information. When the input device 130 is constituted by a touch sensor, the display device 120 and the input device 130 may be constituted as a unified touch panel. The display device 120 and the input device 130 are disposed in a seat on which an occupant sits or in the vicinity thereto.

Through an occupant's operation, various types of information such as authentication information (a user ID and a password), an occupant's height, a scheduled alighting place of an occupant, an agreement with alighting at a recommended alighting place which will be described later, a target walking distance of an occupant, and calorie consumption of an occupant are input to the input device 130.

The fare settling device 140 is a device that is provided in the vicinity of an entrance of the vehicle and settles a fare when an occupant uses the vehicle. The occupant can settle the fare using cash, an electronic season ticket, an electronic ticket, an electronic money card, or the like using the fare settling device 140.

The vehicle control system 160 is a system that automatically controls a vehicle when the vehicle is an automated driving vehicle. The vehicle control system 160 includes a motor or an engine which is a drive source of the vehicle, a steering device, a brake device, a battery, an onboard camera that images surroundings of the vehicle, and a positioning information receiver (global positioning system (GPS) receiver). The vehicle control system 160 includes a control device that controls automated driving of the vehicle based on position information of the vehicle, map information, and an image captured by the onboard camera.

The notification device 170 is a device that notifies a driver of the vehicle of a stop place (a bus stop) at which the vehicle stops, and is provided in the vicinity of a meter panel of a driver's seat when the vehicle is manually driven. The notification device 170 includes an LED. When an occupant pushes an alighting button provided in the vehicle while the vehicle is traveling, the notification device 170 emits light to notify the driver that the vehicle stops at the next stop place. When a recommended alighting place which will be described later is notified by the control device 150, the notification device 170 emits light before a recommended stop place based on the current position of the vehicle acquired from the vehicle control system 160 and map information, and notifies the driver that the vehicle stops at the recommended alighting place. The notification device 170 may include an LCD and a speaker.

The control device 150 is an aspect of a health management device and includes a processor 152, a memory 154, and a communication interface 156. The processor 152 includes one or more central processing units (CPUs) and peripheral circuits thereof. The processor 152 may further include other calculation circuits such as a logic calculation unit, a numerical calculation unit, or a graphic processing unit. The memory 154 includes, for example, a volatile semiconductor memory and a nonvolatile semiconductor memory. The communication interface 156 includes an interface circuit that connects the control device 150 to the onboard network.

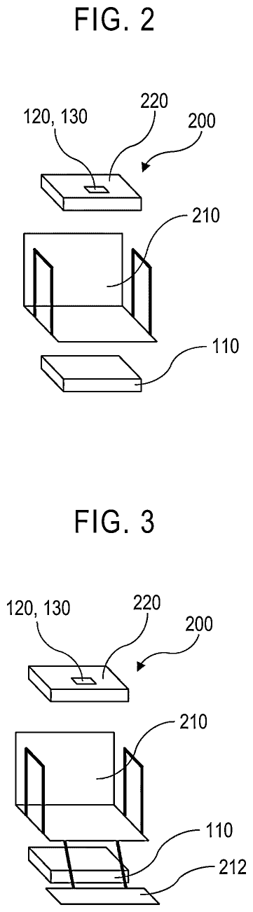

FIGS. 2 and 3 are diagrams schematically illustrating a configuration of a seat 200 on which an occupant sits and which is provided in a vehicle. In the example illustrated in FIG. 2, the seat 200 includes a main body 210 and a cushion 220. The main body 210 includes a seat plate, a backrest, and an armrest, and the cushion 220 is disposed on the seat plate. An occupant sits on the cushion 220.

The main body 210 of the seat 200 is placed on the weight scale 110. When an occupant sits on the seat 200 and legs are detached from the floor, the total weight of the occupant is applied to the weight scale 110, and the weight scale 110 detects a characteristic value associated with a physical body of an occupant such as the weight of the occupant.

In the example illustrated in FIG. 3, the seat 200 includes a main body 210 and a cushion 220, and a footrest 212 extending forward from the main body 210 is additionally provided. Similarly to FIG. 2, the main body 210 includes a seat plate, a backrest, and an armrest, and the cushion 220 is disposed on the seat plate. When an occupant sits on the seat 200 and place feet on the footrest 212, the total weight of the occupant is applied to the weight scale 110, and the weight scale 110 detects a characteristic value associated with a physical body of an occupant such as the weight of the occupant.

In the example illustrated in FIG. 2, when an occupant does not detach the feet from the floor, the weight is not accurately detected. Accordingly, when a weight or the like is measured, a message for prompting an occupant to detach feet from the floor is displayed on the display device 120.

In the examples illustrated in FIGS. 2 and 3, the display device 120 and the input device 130 are provided in a front end portion of a bearing surface of the cushion 220. The weight or the like detected by the weight scale 110 is displayed on the display device 120. Accordingly, the occupant can see the weight or the like displayed on the display device 120. Since the display device 120 is disposed on the bearing surface of the cushion 220 of the seat 200 on which the occupant sits, the occupant can easily ascertain display details. Since the display device 120 is disposed on the bearing surface, the occupant can easily hide the display details and thus the display details on the display device 120 cannot be seen by another occupant and privacy of the occupant can be protected. A peep preventing film may be attached to the screen of the display device 120 such that the display details on the display device 120 cannot be seen by another occupant.

A plurality of seats 200 on which an occupant can sit is provided in the vehicle, and the weight scale 110, the display device 120, and the input device 130 are provided in each of the plurality of seats 200. Accordingly, even when an occupant sits on any seat 200, the occupant can see a measurement result of the weight or the like. The display device 120 and the input device 130 of each seat 200 are correlated with the weight scale 110 of the corresponding seat 200 using a seat number.

The detected characteristic value associated with a physical body of an occupant is stored in the memory 154 of the control device 150. A difference in characteristic value between previous boarding and current boarding is displayed on the display device 120. A service based on the difference in characteristic value is provided to the occupant, and a fare for using the vehicle is lower for a healthier person.

The control device 150 may be provided at a position separated from the seat 200. The devices such as the weight scale 110, the display device 120, the input device 130, the fare settling device 140, and the notification device 170 may be Internet of Things (IoT) devices, and the control device 150 may communicate with the devices via a communication network such as the Internet. In this case, the control device 150 may be provided in a server outside of the vehicle.

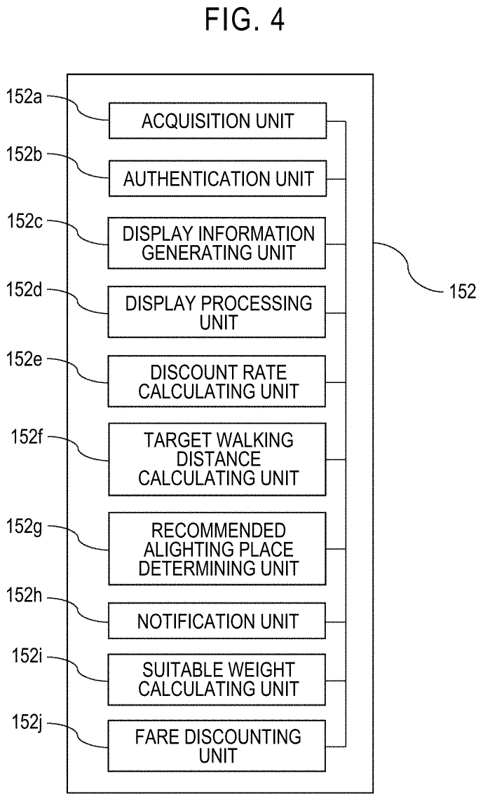

FIG. 4 is a diagram schematically illustrating functional blocks of the processor 152 of the control device 150. The processor 152 of the control device 150 includes an acquisition unit 152a, an authentication unit 152b, a display information generating unit 152c, a display processing unit 152d, a discount rate calculating unit 152e, a target walking distance calculating unit 152f, a recommended alighting place determining unit 152g, a notification unit 152h, a suitable weight calculating unit 152i, and a fare discounting unit 152j. These constituent units of the processor 152 are functional modules which are realized by a computer program operating in the processor 152. That is, the constituent units of the processor 152 are realized by the processor 152 and a program (software) causing the processor 152 to function. The program may be stored in the memory 154 of the control device 150 or a recording medium connected from the outside. Alternatively, the constituent units of the processor 152 may be dedicated calculation circuits provided in the processor 152.

The acquisition unit 152a of the processor 152 acquires a weight of an occupant detected by a detector provided in a seat 200 of a vehicle that transports the occupant or a value that can be detected using the weight as a characteristic value associated with a physical body of the occupant. As described above, the weight scale 110 is a type of a detector. Specifically, when an occupant sits on the seat 200, the acquisition unit 152a acquires a characteristic value detected at the timing at which the occupant sits. The acquisition unit 152a acquires a seat number of the weight scale 110 having detected the characteristic value along with the characteristic value. The acquisition unit 152a acquires a characteristic value which was detected in the past and stored in the memory 154 from the memory 154.

The acquisition unit 152a acquires various types of information such as authentication information, an occupant's height, a scheduled alighting place of the occupant, an agreement with alighting at a recommended alighting place, a target walking distance of the occupant, and calorie consumption of the occupant which are input to the input device 130 by the occupant. The acquisition unit 152a acquires the seat number of the input device 130 to which the authentication information has been input along with the various types of information.

Since the characteristic value associated with a physical body of an occupant such as the weight detected by the weight scale 110 may vary due to acceleration or deceleration of the vehicle, the acquisition unit 152a may acquire the characteristic value detected by the weight scale 110 at the timing at which the vehicle stops. The acquisition unit 152a may acquire a constant value when the characteristic value reaches the constant value based on variation of the characteristic value detected by the weight scale 110. Accordingly, the characteristic value is more accurately acquired. The characteristic value is stored in the memory 154 in correlation with authentication information.

The authentication unit 152b of the processor 152 authenticates the occupant based on authentication information. Occupants using the health management system 100 register authentication information along with names thereof in the control device 150 in advance. The registered information such as the authentication information and the names is stored in the memory 154 of the control device 150. When an occupant having registered the authentication information boards the vehicle and uses the health management system 100, first, the occupant inputs authentication information to the input device 130. The authentication unit 152b authenticates the occupant by comparing the authentication information acquired by the acquisition unit 152a with the authentication information stored in the memory 154. When the authentication information acquired by the acquisition unit 152a matches the authentication information stored in the memory 154, authentication in the authentication unit 152b succeeds. On the other hand, when the authentication information acquired by the acquisition unit 152a does not match the authentication information stored in the memory 154, authentication in the authentication unit 152b fails. In this case, the occupant cannot use the health management system 100. When the authentication information is registered, heights of the occupants may be stored in the memory 154 in correlation with the authentication information.

The display information generating unit 152c of the processor 152 generates display information which is displayed on the display device 120 which can be seen by an occupant when the occupant sits on the seat 200 based on the characteristic value and generates the display information which varies according to a difference between the characteristic value detected at the timing at which the occupant sits on the seat 200 and the characteristic value of the occupant detected in the past.

Specifically, when authentication in the authentication unit 152b succeeds, the display information generating unit 152c generates display information which varies according to a difference between the characteristic value detected at the timing at which the authenticated occupant sits on the seat 200 and the characteristic value of the occupant detected in the past based on the authentication information. The acquisition unit 152a acquires characteristic values detected by a plurality of weight scales 110, and the weight scale 110 having detected the characteristic value of the authenticated occupant is identified based on the seat number of the input device 130 to which the authenticated occupant is input. Since the past characteristic values stored in the memory 154 are correlated with the authentication information, the acquisition unit 152a can acquire the characteristic value detected in the past from the memory 154 based on the authentication information. Accordingly, the display information generating unit 152c can generate display information based on the characteristic value detected at the timing at which the occupant sits on the seat 200 and the characteristic value of the occupant detected in the past.

The display information generating unit 152c generates display information indicating the characteristic value detected at the timing at which the occupant sits on the seat 200, the characteristic value of the occupant detected in the past and stored in the memory 154, a suitable value, and a difference between the characteristic value detected at the timing at which the occupant sits on the seat 200 and the suitable value. The display information generating unit 152c generates display information indicating a discount rate, a target walking distance, a recommended alighting place, or the like which will be described later.

The display processing unit 152d of the processor 152 performs a processing of sending the display information generated by the display information generating unit 152c to the display device 120 and displaying display information on the display device 120. More specifically, the display processing unit 152d sends display information to the display device 120 corresponding to a seat number of the weight scale 110 having detected the characteristic value of the authenticated occupant based on the seat number, and displays the display information on the display device 120. Accordingly, it is possible to curb displaying the display information on the display device 120 of a seat other than the seat 200 of the authenticated occupant.

The discount rate calculating unit 152e of the processor 152 calculates a discount rate of a fare for allowing an occupant to use the vehicle based on the difference in characteristic value. The discount rate calculating unit 152e calculates the discount rate such that the discount rate becomes larger as the characteristic value becomes closer to the suitable value for the occupant. The discount rate calculating unit 152e sets the discount rate to zero when the characteristic value does not become closer to the suitable value. Accordingly, in this case, the fare is not discounted.

The target walking distance calculating unit 152f of the processor 152 calculates a target walking distance of the occupant based on the difference in characteristic value. The target walking distance calculating unit 152f calculates the target walking distance such that the target walking distance becomes smaller as the characteristic value becomes closer to the suitable value for the occupant. The target walking distance may be an arbitrary value which is input from the input device 130 by the occupant. The target walking distance calculating unit 152f may calculate the target walking distance such that the target walking distance becomes larger as calorie consumption becomes smaller in consideration of the calorie consumption input from the input device 130 by the occupant.

The recommended alighting place determining unit 152g of the processor 152 determines a recommended alighting place prior by the target walking distance to a scheduled alighting place at which the occupant is scheduled to alight from the vehicle based on the target walking distance and the scheduled alighting place.

When the acquisition unit 152a acquires an agreement with alighting at the recommended alighting place from the occupant, the discount rate calculating unit 152e calculates the discount rate of a fare for allowing the occupant to use the vehicle based on the target walking distance. The discount rate calculating unit 152e calculates the discount rate such that the discount rate becomes larger as the target walking distance becomes larger.

When the acquisition unit 152a acquires an agreement with alighting at the recommended alighting place from the occupant, the notification unit 152h of the processor 152 notifies the vehicle control system 160 that automatically controls the vehicle or the notification device 170 that notifies a driver of the vehicle of a stop place of the recommended alighting place. When the recommended alighting place is notified of, the vehicle control system 160 stops the vehicle at the recommended alighting place based on a current position of the vehicle and map information. When the recommended alighting place is notified of, the notification device 170 notifies the driver that the vehicle is to stop at the recommended alighting place.

When the characteristic value is a weight, the suitable weight calculating unit 152i of the processor 152 calculates a suitable weight for the occupant as the suitable value based on a height of the occupant. The suitable weight calculating unit 152i calculates the suitable weight from a height of the occupant, for example, using an index such as a BMI value. When the characteristic is a characteristic value other than the weight, the suitable weight calculating unit 152i may calculate a suitable value of the characteristic value.

The fare discounting unit 152j of the processor 152 performs a process of discounting the fare for allowing the occupant to use the vehicle based on the discount rate calculated by the discount rate calculating unit 152e. Specifically, the fare discounting unit 152j sends the discount rate calculated by the discount rate calculating unit 152e to the fare settling device 140, and returns discounted money (cash) corresponding to the discount rate of the fare from the fare settling device 140 to the occupant when the occupant settles the fare. Alternatively, the fare discounting unit 152j gives electronic money corresponding to the discounted money of the fare or an electronic point usable for a point service to an electronic season ticket, an electronic ticket, an electronic money card, or the like which is owned by the occupant via the fare settling device 140.

When the occupant agrees with alighting at the recommended alighting place, the fare to the recommended alighting place is discounted based on the discount rate calculated from the target walking distance and the fare is decreased due to alighting before the scheduled alighting place.

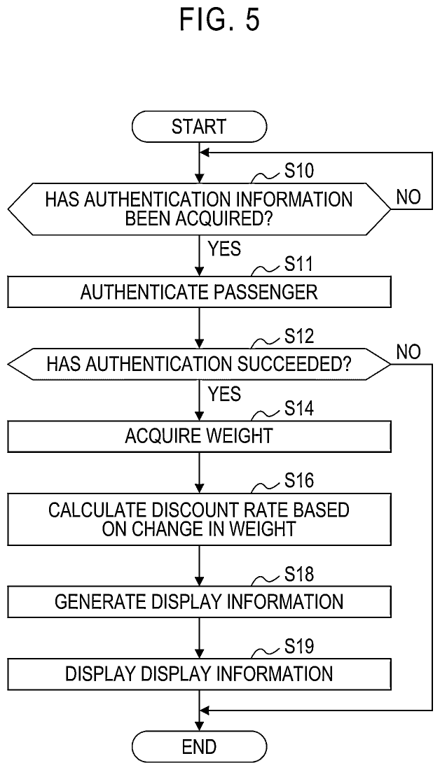

FIG. 5 is a flowchart illustrating a routine which is performed by the processor 152 of the control device 150. The routine illustrated in FIG. 5 is performed at intervals of a predetermined control cycle by the processor 152. In the following description, it is assumed that a characteristic value associated with a physical body of an occupant is a weight. First, it is determined whether the acquisition unit 152a of the processor 152 has acquired authentication information (Step S10). When authentication information has been acquired, the routine proceeds to Step S11. When authentication information has not been acquired, the routine waits in Step S10.

Then, the authentication unit 152b of the processor 152 authenticates an occupant based on the authentication information (Step S11). When the authentication succeeds (YES in Step S12), the acquisition unit 152a of the processor 152 acquires weight detected at the timing at which the occupant sits on a seat 200 (Step S14). In Step S14, the acquisition unit 152a of the processor 152 also acquires a weight detected in the past and stored in the memory 154 from the memory 154. Since the weight is stored in the memory 154 in correlation with the authentication information, the acquisition unit 152a acquires the weight stored in the memory 154 based on the authentication information with which authentication has succeeded. When the authentication fails (NO in Step S12), the routine ends.

Then, the discount rate calculating unit 152e of the processor 152 calculates a discount rate of a fare for allowing the occupant to use the vehicle based on a difference in weight of the occupant (Step S16). The discount rate calculating unit 152e calculates the discount rate such that the discount rate becomes larger as the weight becomes closer to a suitable weight based on a difference between the weight detected at the timing at which the occupant sits on the seat 200 and the weight detected in the past and stored in the memory 154. The suitable weight is calculated based on a height of the occupant input to the input device 130 by the occupant.

Then, the display information generating unit 152c of the processor 152 generates display information indicating the discount rate (Step S18). Then, the display processing unit 152d of the processor 152 sends the display information generated by the display information generating unit 152c to the display device 120 and displays the display information on the display device 120 (Step S19). After Step S19, the routine ends.

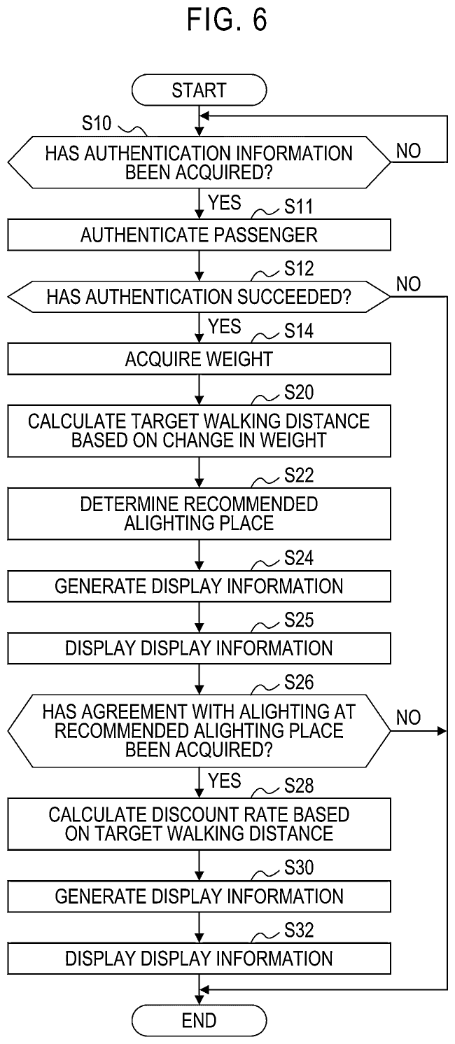

FIG. 6 is a flowchart illustrating another routine which is performed by the processor 152 of the control device 150. The processes of Steps S10 to S14 are the same as illustrated in FIG. 5 and thus the processes of Step S20 and steps subsequent thereto will be described below.

Subsequently to Step S14, the target walking distance calculating unit 152f of the processor 152 calculates a target walking distance of the occupant based on the difference in weight of the occupant (Step S20). The target walking distance calculating unit 152f calculates the target walking distance such that the target walking distance becomes smaller as the weight becomes closer to a suitable weight for the occupant based on the difference between the weight detected at the timing at which the occupant sits on the seat 200 and the weight detected in the past and stored in the memory 154. The suitable weight is calculated based on a height of the occupant input to the input device 130 by the occupant. Then, the recommended alighting place determining unit 152g of the processor 152 determines a recommended alighting place (Step S22). The recommended alighting place determining unit 152g determines the recommended alighting place which is prior by the target walking distance to a scheduled alighting place at which the occupant is scheduled to alight from the vehicle based on the target walking distance and the scheduled alighting place.

Then, the display information generating unit 152c of the processor 152 generates display information indicating the recommended alighting place (Step S24). Then, the display processing unit 152d of the processor 152 sends the display information generated by the display information generating unit 152c to the display device 120 and displays the display information on the display device 120 (Step S25).

Then, it is determined whether the occupant has agreed with alighting at the recommended alighting place (Step S26). Specifically, in Step S26, it is determined whether the acquisition unit 152a of the processor 152 has acquired an agreement with alighting at the recommended alighting place.

When the acquisition unit 152a has acquired an agreement with alighting at the recommended alighting place (YES in Step S26), the discount rate calculating unit 152e calculates a discount rate of a fare for allowing the occupant to use the vehicle based on the target walking distance (Step S28). On the other hand, when the acquisition unit 152a has not acquired an agreement with alighting at the recommended alighting place (NO in Step S26), the routine ends.

Then, the display information generating unit 152c generates display information indicating the discount rate (Step S30). Then, the display processing unit 152d of the processor 152 sends the display information generated by the display information generating unit 152c to the display device 120 and displays the display information on the display device 120 (Step S32). After Step S32, the routine ends.

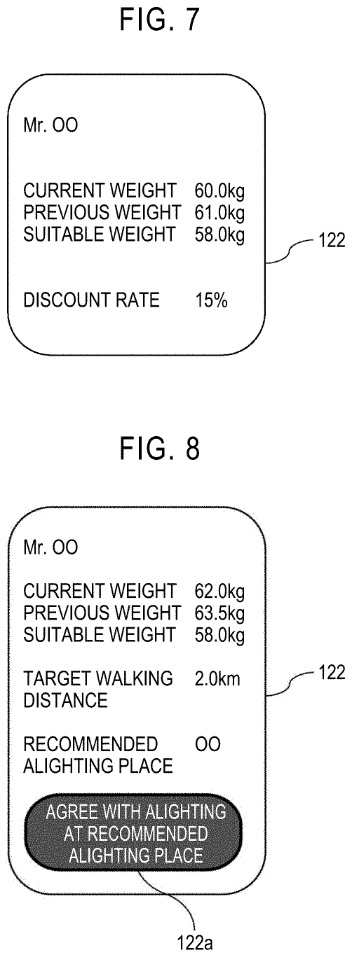

FIGS. 7 and 8 are diagrams schematically illustrating a display screen 122 of the display device 120 and are diagrams illustrating a situation in which display information is displayed on the display screen 122. In the example illustrated in FIG. 7, a weight detected at the timing at which an occupant sits on a seat 200 (a current weight), a weight detected in the past and stored in the memory 154 (a previous weight), a suitable weight calculated based on a height of the occupant, and a discount rate of a fare are displayed on the display screen 122. In this case, since the weight detected at the timing at which the occupant sits on the seat 200 (is 60.0 kg, the weight detected in the past is 61.0 kg, and the suitable weight is 58.0 kg, the difference between the weight detected at the timing at which the occupant sits on the seat 200 and the weight of the occupant detected in the past is a change approaching the suitable weight. Accordingly, the discount rate such that the discount rate becomes larger as the weight becomes closer to the suitable weight is calculated and is displayed on the display screen of the display device 120.

In the example illustrated in FIG. 8, the current weight, the previous weight, the suitable weight, the target walking distance, and the recommended alighting place are displayed on the display screen 122. In the example illustrated in FIG. 8, when the display device 120 and the input device 130 are configured as a unified touch panel, a button 122a for allowing the occupant to input an agreement with alighting at the recommended alighting place is displayed on the display screen 122. When the occupant pushes the button 122a, an agreement with alighting at the recommended alighting place is input to the input device 130. When the processor 152 acquires the agreement with alighting at the recommended alighting place, the discount rate of the fare is calculated, and the discount rate is displayed on the display screen 122 similarly to FIG. 7.

As described above, according to the first embodiment, display information which varies according to a difference between a characteristic value associated with a physical body of an occupant and detected at the timing at which the occupant sits on the seat 200 and the characteristic value of the occupant detected in the past is generated. Specifically, a discount rate of a fare which varies according to the difference in characteristic value is calculated or a recommended alighting place which varies according to the difference in characteristic value is determined, and the discount rate or the recommended alighting place is displayed on the display device 120. Accordingly, since the occupant having seen the display information has an intention such that the characteristic value changes better, it is possible to promote health management of the occupant and to use an opportunity when boarding the vehicle for health promotion for the occupant.

### 2. Second Embodiment

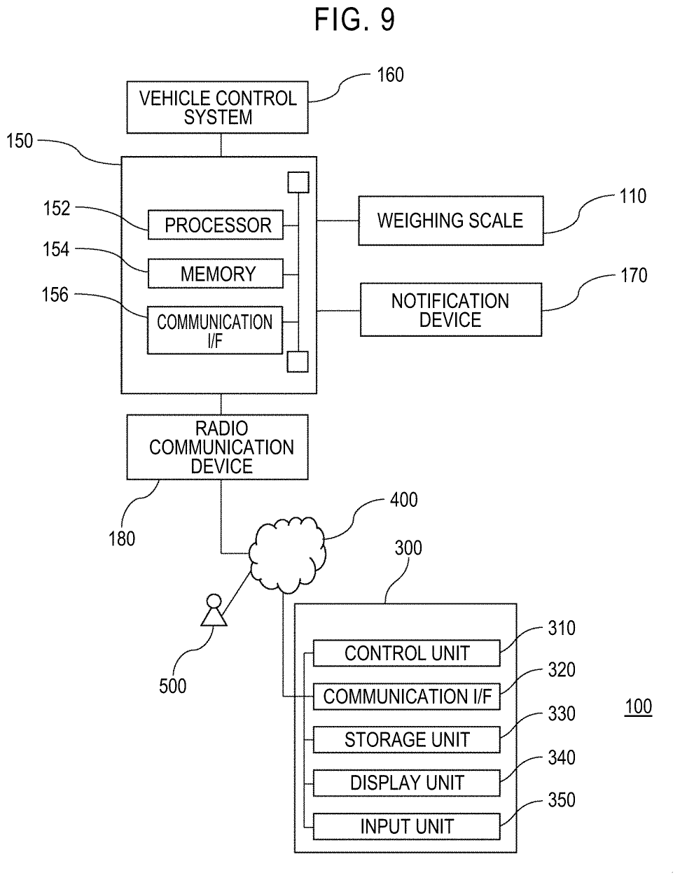

FIG. 9 is a diagram schematically illustrating a configuration of a health management system 100 according to a second embodiment of the disclosure. The health management system 100 according to the second embodiment includes a mobile terminal 300 owned by an occupant in addition to the configuration according to the first embodiment. The control device 150 and the mobile terminal 300 are connected to communicate with each other via a communication network 400 such as the Internet and a radio base station 500. Accordingly, a radio communication device 180 is connected to the control device 150. The control device 150 and the mobile terminal 300 may be connected by radio communication such as WiFi, a radio communication network of a mobile phone network such as LTE, LTE-Advance, 4G, or 5G, a dedicated network such as a virtual private network (VPN), a network such as a LAN, or short-range radio communication based on near field communication (NFC).

In the second embodiment, the weight scale 110, the vehicle control system 160, the control device 150, and the notification device 170 have the same configurations as in the first embodiment.

The radio communication device 180 includes, for example, an antenna and a signal processing circuit that performs various processes associated with radio communication such as modulation and demodulation of radio signals. The radio communication device 180 receives a downlink radio signal from the radio base station 500 and transmits an uplink radio signal to the radio base station 500. That is, the radio communication device 180 takes a signal which is transmitted from the mobile terminal 300 (for example, authentication information, a height of an occupant, a scheduled alighting place of an occupant, an agreement with alighting at a recommended alighting place, a target walking distance of an occupant, calorie consumption of an occupant, and a seat number) from the downlink radio signal received from the radio base station 500 and sends the taken signal to the control device 150. The radio communication device 180 generates an uplink radio signal including a signal (for example, display information and an inquiry about a seat number) received from the control device 150 and transmitted to the mobile terminal 300 and transmits the radio signal.

The mobile terminal 300 includes a control unit 310, a communication interface 320, a storage unit 330, a display unit (display device) 340, and an input unit 350.

The control unit 310 of the mobile terminal 300 is constituted by a processor. The processor includes one or more central processing units (CPUs) and peripheral circuits thereof. The processor may further include other calculation circuits such as a logic calculation unit, a numerical calculation unit, or a graphic processing unit. The control unit 310 provides a function corresponding to a predetermined purpose by executing a computer program executably loaded to a work area of the storage unit 330.

The communication interface 320 of the mobile terminal 300 is a communication interface with the communication network 400 and the radio base station 500 and includes, for example, an antenna and a signal processing circuit that performs various processes associated with radio communication such as modulation and demodulation of radio signals. The communication interface 320 receives a downlink radio signal from the radio base station 500 connected to the communication network 400 and transmits an uplink radio signal to the radio base station 500. The communication interface 320 takes a signal which is transmitted from the control device 150 to the mobile terminal 300 from the received downlink radio signal and sends the taken signal to the control unit 310. The communication interface 320 generates an uplink radio signal including a signal received from the control unit 310 and transmitted to the control device 150 and transmits the radio signal.

The storage unit 330 of the mobile terminal 300 includes, for example, a volatile semiconductor memory and a nonvolatile semiconductor memory. The display unit 340 of the mobile terminal 300 includes, for example, a liquid crystal display (LCD) and displays various types of information. The input unit 350 of the mobile terminal 300 includes, for example, a touch sensor and a keyboard, and information corresponding to a user's operation is input to the input unit 350. When the input unit 350 includes a touch sensor, the display unit 340 and the input unit 350 may be configured as a unified touch panel.

In the second embodiment, various types of information such as a weight of an occupant are transmitted from the control device 150 to the mobile terminal 300 and are displayed on the display unit 340 of the mobile terminal 300. Various types of information such as authentication information, an occupant's height, a scheduled alighting place of the occupant, an agreement with alighting at a recommended alighting place, a target walking distance of the occupant, and calorie consumption of the occupant are input to the input unit 350 of the mobile terminal 300 and transmitted to the control device 150 by the occupant's operation. Accordingly, the display device 120 and the input device 130 according to the first embodiment may not be provided in the vehicle.

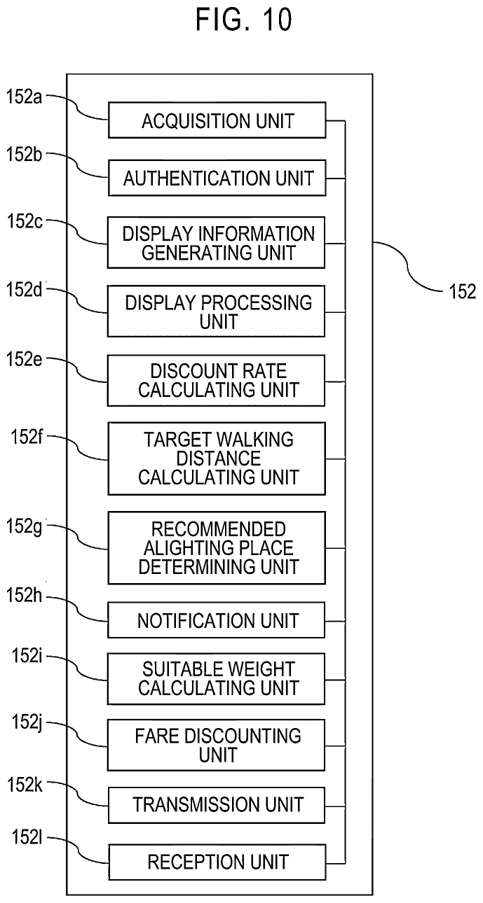

FIG. 10 is a diagram schematically illustrating functional blocks of the processor 152 of the control device 150. The functional blocks of the processor 152 according to the second embodiment include a transmission unit 152k and a reception unit 152l in addition to the functional blocks of the processor 152 according to the first embodiment illustrated in FIG. 4. In FIG. 10, the elements except for the transmission unit 152k and the reception unit 152l are configured in the same way as in the first embodiment.

The transmission unit 152k of the processor 152 transmits display information generated by the display information generating unit 152c, an inquiry about a seat number, and the like to the mobile terminal 300. The transmission unit 152k transmits the display information and the like to the mobile terminal 300 via the radio communication device 180.

The reception unit 152l of the processor 152 receives various types of information such as an occupant's height, a scheduled alighting place of the occupant, an agreement with alighting at a recommended alighting place, a target walking distance of the occupant, calorie consumption of the occupant, and a seat number which are transmitted by the communication interface 320 of the mobile terminal 300. The reception unit 152l receives such various types of information via the radio communication device 180. The acquisition unit 152a of the processor 152 acquires the various types of information received by the reception unit 152l.

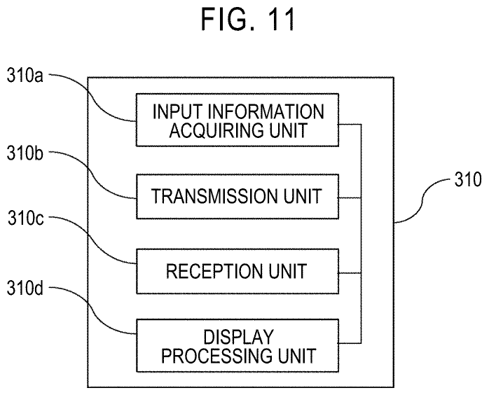

FIG. 11 is a diagram schematically illustrating functional blocks of the control unit 310 of the mobile terminal 300. The control unit 310 of the mobile terminal 300 includes an input information acquiring unit 310a, a transmission unit 310b, a reception unit 310c, and a display processing unit 310d. These constituents of the control unit 310 are, for example, functional modules which are realized by a computer program operating in the control unit 310. That is, the constituents of the control unit 310 are realized by the control unit 310 and a program (software) causing the control unit 310 to function. The program may be stored in the storage unit 330 of the mobile terminal 300 or a recording medium connected from the outside. Alternatively, the constituents of the control unit 310 may be a dedicated calculation circuit provided in the control unit 310.

The input information acquiring unit 310a of the control unit 310 of the mobile terminal 300 acquires input information which is input by allowing a user to operate the input unit 350. The input information includes various types of information such as authentication information, an occupant's height, a scheduled alighting place of the occupant, an agreement with alighting at a recommended alighting place, a target walking distance of the occupant, calorie consumption of the occupant, and a seat number.

The transmission unit 310b of the control unit 310 performs a process of transmitting various types of information acquired by the input information acquiring unit 310a to the control device 150 via the communication interface 320. When the mobile terminal 300 has a function of detecting a walking distance or the like and a function of calculating calorie consumption of an occupant based on the walking distance, the transmission unit 310b may transmit the calculated calorie consumption to the control device 150.

The reception unit 310c of the control unit 310 performs a process of receiving display information, an inquiry about a seat number, and the like transmitted from the control device 150 via the communication interface 320. The display processing unit 310d of the control unit 310 performs a process of sending the display information, the inquiry about a seat number, and the like received by the reception unit 310c to the display unit 340 and displaying the display information on the display unit 340.

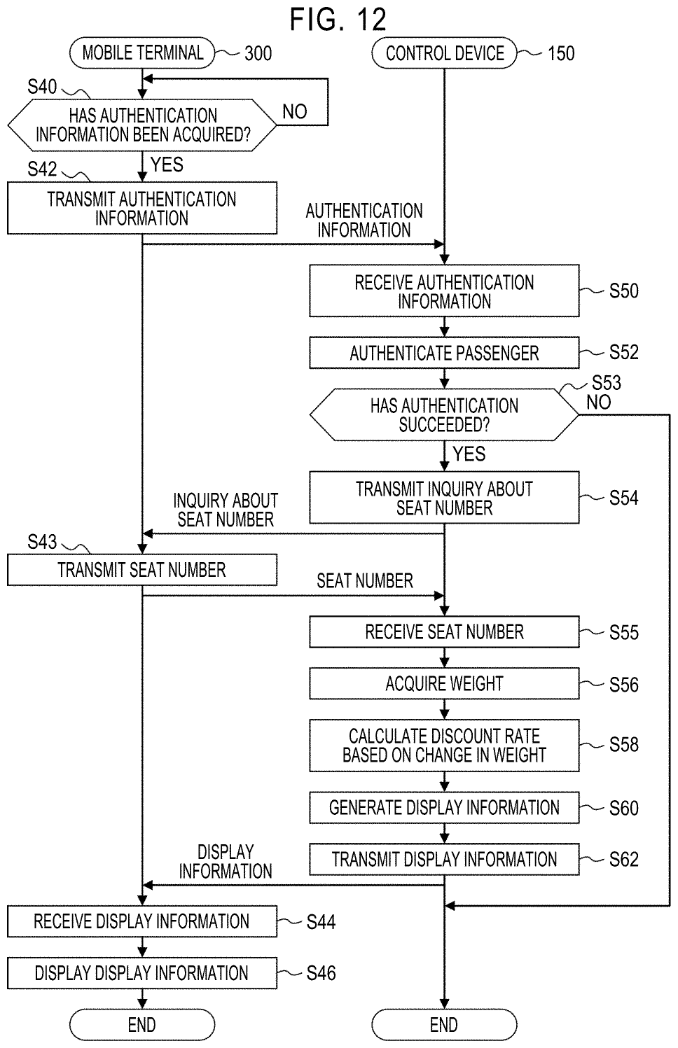

FIG. 12 is a sequence diagram illustrating a routine which is performed by the mobile terminal 300 and the control device 150. The routine illustrated in FIG. 12 is performed by the control unit 310 of the mobile terminal 300 and the processor 152 of the control device 150.

First, when an occupant sits on a seat 200, it is determined whether the input information acquiring unit 310a of the control unit 310 of the mobile terminal 300 has acquired authentication information which is input by allowing the occupant to operate the input unit 350 (Step S40). When authentication information has been acquired, the routine proceeds to Step S42. When authentication information has not been acquired, the routine waits in Step S42. In Step S42, the transmission unit 310b of the control unit 310 transmits the authentication information to the control device 150.

Then, the reception unit 152l of the processor 152 of the control device 150 receives the authentication information from the mobile terminal 300 (Step S50), and the acquisition unit 152a acquires the authentication information. Then, the authentication unit 152b of the processor 152 authenticates the occupant based on the authentication information (Step S52), and when authentication succeeds (YES in Step S53), the transmission unit 152k of the processor 152 transmits an inquiry about a seat number to the mobile terminal 300 (Step S54).

In the mobile terminal 300, when the inquiry about a seat number is received, the inquiry about a seat number is displayed on the display unit 340. When a user having seen the inquiry about a seat number displayed on the display unit 340 inputs a seat number from the input unit 350, the input information acquiring unit 310a acquires the seat number and the transmission unit 310b transmits the seat number to the control device 150 (Step S43).

In the control device 150, when the reception unit 152l of the processor 152 receives the seat number (Step S55), the acquisition unit 152a of the processor 152 acquires a weight detected at the timing at which the occupant sits on the seat 200 corresponding to the received seat number (Step S56). In Step S56, the acquisition unit 152a of the processor 152 acquires a weight detected in the past and stored in the memory 154 from the memory 154. Since the weight is stored in the memory 154 in correlation with authentication information, the acquisition unit 152a acquires the weight stored in the memory 154 based on the authentication information with which authentication has succeeded. When the authentication fails (NO in Step S53), the routine ends.

When the control device 150 and the mobile terminal 300 are connected by short-range radio communication based on NFC, the radio communication device 180 is configured as a reader writer that performs short-range radio communication with the mobile terminal 300 and is provided in each seat 200 of the vehicle. In this case, a reader writer of each seat 200 is correlated with seat numbers of the seats 200, and the seat 200 on which the occupant sits is identified based on the seat number correlated with the reader writer when the reader writer receives the authentication information from the mobile terminal 300. Accordingly, in this case, an inquiry about a seat number does not need to be transmitted from the control device 150 to the mobile terminal 300.

Then, the discount rate calculating unit 152e of the processor 152 calculates a discount rate of a fare for allowing the occupant to use the vehicle based on a difference in weight of the occupant (Step S58). The discount rate calculating unit 152e calculates the discount rate such that the discount rate becomes larger as the weight becomes closer to a suitable weight for the occupant based on a difference in weight between the weight detected at the timing at which the occupant sits on the seat 200 and the weight detected in the past and stored in the memory 154. The suitable weight is calculated based on a height of the occupant received from the mobile terminal 300.

Then, the display information generating unit 152c of the processor 152 generates display information indicating the discount rate (Step S60). Then, the transmission unit 152k of the processor 152 transmits the display information generated by the display information generating unit 152c to the mobile terminal 300 (Step S62).

In the mobile terminal 300, the reception unit 310c of the control unit 310 receives the display information transmitted from the control device 150 (Step S44). Then, the display processing unit 310d of the control unit 310 sends the display information received from the control device 150 to the display unit 340 and displays the display information on the display unit 340 (Step S46). After Step S46, the routine ends.

In FIG. 12, the routine corresponding to FIG. 5 according to the first embodiment is illustrated as the routine which is performed by the processor 152 of the control device 150, but the routine which is performed by the processor 152 may be the routine corresponding to FIG. 6.

Similarly to FIGS. 7 and 8 according to the first embodiment, the display information is displayed on the display unit 340 of the mobile terminal 300.

### Modified Example

A weight of an occupant detected by the weight scale 110 may be transmitted to the mobile terminal 300 and stored in the storage unit 330 of the mobile terminal 300. In this case, when the occupant is authenticated, the transmission unit 310b of the mobile terminal 300 transmits the weight detected in the past and stored in the storage unit 330 to the control device 150. The reception unit 152l of the processor 152 of the control device 150 receives the weight detected in the past and transmitted from the mobile terminal 300, and the acquisition unit 152a of the processor 152 acquires the weight. The display information generating unit 152c of the processor 152 generates display information which varies according to a difference in weight between the weight detected at the timing at which the occupant sits on the seat 200 and the weight detected in the past and acquired from the mobile terminal 300.

As described above, according to the second embodiment, a characteristic value associated with a physical body of an occupant is transmitted to the mobile terminal 300 owned by the occupant, and display information which varies according to change of the characteristic value is generated. Accordingly, it is possible to prevent another person from seeing the characteristic value and to protect privacy of the occupant.

What is claimed is:

1. A health management device comprising:
an acquisition unit configured to acquire a weight of an occupant detected by a detector provided in a seat of a vehicle that transports the occupant or a value detectable using the weight as a characteristic value associated with a physical body of the occupant; and
a display information generating unit configured to generate display information which is displayed on a display device which is visible to an occupant when the occupant sits on the seat based on the characteristic value, the display information varying according to a difference between the characteristic value detected at the timing at which the occupant sits on the seat and the characteristic value of the occupant detected in the past.

2. The health management device according to claim 1, further comprising a discount rate calculating unit configured to calculate a discount rate of a fare for allowing an occupant to use the vehicle based on the difference in characteristic value,
wherein the display information generating unit is configured to generate the display information indicating the discount rate.

3. The health management device according to claim 2, wherein the discount rate calculating unit is configured to calculate the discount rate such that the discount rate becomes larger as the characteristic value becomes closer to a suitable value for the occupant.

4. The health management device according to claim 1, further comprising:
a target walking distance calculating unit configured to calculate a target walking distance of an occupant based on the difference in characteristic value; and
a recommended alighting place determining unit configured to determine a recommended alighting place which is the target walking distance prior to a scheduled alighting place based on the target walking distance and the scheduled alighting place at which the occupant is scheduled to alight,
wherein the display information generating unit is configured to generate display information indicating the recommended alighting place.

5. The health management device according to claim 4, wherein the target walking distance calculating unit is configured to calculate the target walking distance such that the target walking distance becomes smaller as the characteristic value becomes closer to a suitable value for the occupant.

6. The health management device according to claim 4, further comprising a discount rate calculating unit configured to calculate a discount rate of a fare for allowing an occupant to use the vehicle based on the target walking distance when an agreement with alighting at the recommended alighting place is acquired from the occupant, and
wherein the display information generating unit is configured to generate display information indicating the discount rate.

7. The health management device according to claim 6, wherein the discount rate calculating unit is configured to calculate the discount rate such that the discount rate becomes larger as the target walking distance becomes larger.

8. The health management device according to claim 4, further comprising a notification unit configured to notify a control system that automatically controls the vehicle or a notification device that notifies a driver of the vehicle of a stop place of the recommended alighting place when an agreement with alighting at the recommended alighting place is acquired from the occupant.

9. The health management device according to claim 3, wherein the characteristic value is a weight, and
wherein the health management device further comprises a suitable weight calculating unit configured to calculate a suitable weight as the suitable value based on a height of the occupant.

10. The health management device according to claim 1, wherein the display device is provided in a front end portion of a bearing surface of the seat.

11. The health management device according to claim 1, further comprising a display processing unit configured to display the display information on the display device provided in the seat or in the vicinity of the seat.

12. The health management device according to claim 1, wherein the display device is provided in a mobile terminal that is carried by an occupant, and
wherein the health management device further comprises a transmission unit configured to transmit the display information to the mobile terminal.

13. The health management device according to claim 1, further comprising an authentication unit configured to authenticate an occupant based on authentication information,
wherein the display information generating unit is configured to generate the display information which varies according to the difference in characteristic value of the occupant of which authentication has succeeded.

14. The health management device according to claim 13, further comprising a memory configured to store the characteristic value,
wherein the characteristic value is stored in the memory in correlation with the authentication information, and
wherein the acquisition unit is configured to acquire the characteristic value detected in the past from the memory based on the authentication information.

15. The health management device according to claim 13, wherein the authentication information is acquired from an input device to which an occupant is able to input information and which is provided in the vicinity of the seat or a mobile terminal that is carried by the occupant.

16. The health management device according to claim 1, wherein the characteristic value is at least one of a weight, a body fat percentage, a visceral fat level, a subcutaneous fat percentage, a basal metabolic rate, a skeletal muscle percentage, a bone mass, a muscle mass, a BMI, and an FFMI.

17. A health management method comprising:
a step of acquiring a weight of an occupant detected by a detector provided in a seat of a vehicle that transports the occupant or a value detectable using the weight as a characteristic value associated with a physical body of the occupant; and
a step of generating display information which is displayed on a display device which is visible to an occupant when the occupant sits on the seat based on the characteristic value, the display information varying according to a difference between the characteristic value detected at the timing at which the occupant sits on the seat and the characteristic value of the occupant detected in the past.

18. A health management system in which a health management device and a mobile terminal are connected in a radio communication manner,
wherein the health management device includes: an acquisition unit configured to acquire a weight of an occupant detected by a detector provided in a seat of a vehicle that transports the occupant or a value detectable using the weight as a characteristic value associated with a physical body of the occupant; a display information generating unit configured to generate display information which is displayed on a display device which is visible to an occupant when the occupant sits on the seat based on the characteristic value, the display information varying according to a difference between the characteristic value detected at the timing at which the occupant sits on the seat and the characteristic value of the occupant detected in the past; and a transmission unit configured to transmit the display information to the mobile terminal, and
wherein the mobile terminal includes a reception unit configured to receive the display information and a display processing unit configured to display the display information on a display unit provided in the mobile terminal.
